(19) United States
(12) Patent Application Publication
(10) Pub. No.: US 2007/0190180 A1
(43) Pub. Date: Aug. 16, 2007

(54) Methods of treating cancer with high potency lipid-based platinum compound formulations administered intravenously

(76) Inventors: Frank G. Pilkiewicz, Princeton, NJ (US); Roman Perez-Soler, New York, NY (US); Walter Perkins, Pennington, NJ (US); Yiyu Zou, Bronx, NY (US); Mary E. Neville, Jamesburg, NJ (US); Jin K. Lee, Belle Mead, NJ (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/592,714

(22) Filed: Nov. 3, 2006

Related U.S. Application Data
(60) Provisional application No. 60/734,491, filed on Nov. 8, 2005.

(51) Int. Cl. A61K 33/24; A61K 31/555; A61K 31/282

(52) U.S. Cl. 424/649; 514/184; 514/492

(57) ABSTRACT

In one aspect, the present invention relates to methods of treating cancer in a patient comprising administering intravenously to a patient in need thereof a cancer treating effective amount of a lipid-based platinum compound formulation wherein the concentration of the platinum compound of the lipid-based platinum compound formulation is greater than about 1.2 mg/ml.

### RELATED APPLICATIONS

This application claims the benefit of priority to U.S. Provisional Patent Application Ser. No. 60/734,491, filed Nov. 8, 2005, which is hereby incorporated by reference in its entirety.

### INTRODUCTION

Parenteral routes of administration involve injections into various compartments of the body. Parenteral routes include intravenous (iv), i.e. administration directly into the vascular system through a vein; intra-arterial (ia), i.e. administration directly into the vascular system through an artery; intraperitoneal (ip), i.e. administration into the abdominal cavity; subcutaneous (sc), i.e. administration under the skin; intramuscular (im), i.e. administration into a muscle; and intradermal (id), i.e. administration between layers of skin. The parenteral route is preferred over oral ones in many occurrences. For example, when the drug to be administered would partially or totally degrade in the gastrointestinal tract, parenteral administration is preferred. Similarly, where there is need for rapid response in emergency cases, parenteral administration is usually preferred over oral.

Regional delivery of chemotherapy into the vascular system through a vein via iv administration has been found to be a safe and effective treatment for locally recurrent cancers such as, for example, liver, spleen, and lung cancer.

Cisplatin—cis-diamine-dichloroplatinum (II)—is one of the more effective anti-tumor agents used in the systemic treatment of cancers. This chemotherapeutic drug is highly effective in the treatment of tumor models in laboratory animals and in human tumors, such as endometrial, bladder, ovarian and testicular neoplasms, as well as squamous cell carcinoma of the head and neck (Sur, et al., 1983 Oncology 40(5): 372-376; Steerenberg, et al., 1988 Cancer Chemother Pharmacol. 21(4): 299-307). Cisplatin is also used extensively in the treatment of lung carcinoma, both SCLC and NSCLC (Schiller et al., 2001 Oncology 61(Suppl 1): 3-13). It is typically administered intravenously as an aqueous solution, either as a bolus injection or via infusion over a number of hours. It has been marketed both as a lyophilized powder for reconstitution into an aqueous solution, or as a ready-to-use aqueous solution. Intravenous delivery typically requires an aqueous solution, or, in some cases, a liquid emulsion or liposome system, wherein solid particulates which may occlude vessels and capillaries are absent. The concentration of cisplatin in such intravenously injectable solutions is limited by its solubility in water, on the order of 1 mg/ml. Other active platinum compounds (defined below) are also useful in cancer treatment.

Like other cancer chemotherapeutic agents, active platinum compounds such as cisplatin are typically highly toxic. The main disadvantages of cisplatin are its extreme nephrotoxicity, which is the main dose-limiting factor, its rapid excretion via the kidneys, with a circulation half life of only a few minutes, and its strong affinity to plasma proteins (Freise, et al., 1982 Arch Int Pharmacodyn Ther. 258(2): 180-192).

Attempts to minimize the toxicity of active platinum compounds have included combination chemotherapy, synthesis of analogues (Prestayko et al., 1979 Cancer Treat Rev. 6(1): 17-39; Weiss, et al., 1993 Drugs. 46(3): 360-377), immunotherapy and entrapment in liposomes (Sur, et al., 1983; Weiss, et al., 1993). Antineoplastic agents, including cisplatin, entrapped in liposomes have a reduced toxicity, relative to the agent in free form, while retaining antitumor activity (Steerenberg, et al., 1987; Weiss, et al., 1993).

Cisplatin, however, is difficult to efficiently entrap in liposomes or lipid complexes because of the bioactive agent's low aqueous solubility, approximately 1.0 mg/ml at room temperature, and low lipophilicity, both of which properties contribute to a low bioactive agent/lipid ratio.

Liposomes and lipid complexes containing cisplatin suffer from another problem—stability of the composition. In particular, maintenance of bioactive agent potency and retention of the bioactive agent in the liposome during storage are recognized problems (Freise, et al., 1982; Gondal, et al., 1993; Potkul, et al., 1991 Am J Obstet Gynecol. 164(2): 652-658; Steerenberg, et al., 1988; Weiss, et al., 1993) and a limited shelf life of liposomes containing cisplatin, on the order of several weeks at 4° C., has been reported (Gondal, et al., 1993 Eur J Cancer. 29A(11): 1536-1542; Potkul, et al., 1991).

U.S. Pat. No. 6,511,676 to Boulikas discloses iv administration of liposome encapsulated positively charged species such as the aquated form of cisplatin for the treatment of cancer. However, the species are limited to positively charged species and the liposomes require the use of anionic lipids. The liposomes are also further limited by requiring a different lipid composition between their inner and outer membrane bilayers.

Despite the advances made with iv administration of platinum compounds, the dose limiting toxicity and low drug level in targeted tissues of platinum compounds make most therapies fail to improve patients' life-expectancy. It would be advantageous to develop a platinum compound formulation with potency higher than its aqueous solubility limit at room temperature. High potency lipid based platinum compound formulations would reduce the liquid dose volume for a given dose thus reducing the dosing time (duration) as well.

### SUMMARY OF THE INVENTION

It is an object of the present invention to provide a method of treating cancer comprising administering platinum compounds as part of a high potency lipid-based formulation with lower sub-acute toxicity than when the platinum compound is administered without the lipid formulation.

It is also an object of the present invention to provide a high potency lipid-based platinum compound formulation wherein the potency is higher than the aqueous solubility of the platinum compound at room temperature.

It is also an object of the present invention to treat cancer using a high potency lipid-based platinum compound formulation to reduce the volume of formulation that has to be administered to achieve the same level of effectiveness.

It is also an object of the present invention to treat cancer by introducing platinum compounds as high potency lipid based formulations regionally to bypass gastrointestinal degradation that is often associated with oral administration.

The subject invention results from the realization that high potency lipid-based platinum formulations presented herein can be effectively administered intravenously.

In one embodiment, the present invention relates to a method of treating cancer in a patient comprising administering intravenously to a patient in need thereof a cancer treating effective amount of a lipid-based platinum compound formulation wherein the concentration of the platinum compound of the lipid-based platinum compound formulation is greater than about 1.2 mg/ml. In a further embodiment the platinum compound concentration is about 3 mg/ml. In a further embodiment the platinum compound concentration is about 5 mg/ml.

In a further embodiment the present invention relates to the aforementioned method, wherein the platinum compound is selected from the group consisting of: cisplatin, carboplatin (diammine(1,1-cyclobutanedicarboxylato)-platinum(II)), tetraplatin (ormaplatin) (tetrachloro(1,2-cyclohexanediamine-N,N′)-platinum(IV)), thioplatin (bis(O-ethyldithiocarbonato)platinum(II)), satraplatin, nedaplatin, oxaliplatin, heptaplatin, iproplatin, transplatin, lobaplatin, cis-aminedichloro(2-methylpyridine) platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine) platinum(IV)), JM335 (trans-amminedichloro(cyclohexylamine)dihydroxoplatinum(IV)), (trans, trans, trans)bis-mu-(hexane-1,6-diamine)-mu-[diamine-platinum(II)]bis[diamine(chloro) platinum(II)]tetrachloride, and mixture thereof. In a further embodiment, the platinum compound is cisplatin.

In a further embodiment the present invention relates to the aforementioned method, wherein the lipid in the lipid-based platinum compound formulation is comprised of a member selected from the group consisting of: egg phosphatidyl choline (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (PIs), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof. In a further embodiment, the lipid in the lipid-based platinum compound formulation is a mixture of a phospholipid and a sterol. In a further embodiment, the lipid in the lipid-based platinum compound formulation is a mixture of DPPC and cholesterol. In a further embodiment, the lipid in the lipid-based platinum compound formulation is a mixture of DPPC from 50 to 65 mol % and cholesterol from 35 to 50 mol %.

In a further embodiment the present invention relates to the aforementioned method, wherein the cancer is selected from the following: melanoma, testis (germ cell), osteosarcoma, soft tissue sarcoma, thyroid cancer, colon cancer, ovarian cancer, cancer of the kidney, breast cancer, colorectal cancer, prostate cancer, bladder cancer, uterine cancer, lung cancer, stomach cancer, liver cancer, spleen cancer, endometrial, or squamous cell carcinomas of the head and neck. In a further embodiment the cancer is lung, spleen, or liver cancer.

In a further embodiment the present invention relates to the aforementioned method, wherein the ratio of platinum compound to lipid in the lipid-based platinum compound formulation is between 1:5 by weight and 1:50 by weight. In a further embodiment, the lipid-based platinum compound formulation comprises liposomes having a mean diameter of 0.01 microns to 3.0 microns.

In a further embodiment the present invention relates to the aforementioned method, wherein the lipid is a mixture of DPPC and cholesterol, the ratio of platinum compound to lipid in the lipid-based platinum compound formulation is between 1:5 by weight and 1:50 by weight, and wherein the lipid-based platinum compound formulation comprises liposomes having a mean diameter of 0.01 microns to 3.0 microns.

In a further embodiment the present invention relates to the aforementioned method, wherein the lipid is a mixture of DPPC and cholesterol, the ratio of platinum compound to lipid in the lipid-based platinum compound formulation is between 1:5 by weight and 1:50 by weight, the lipid-based platinum compound formulation comprises liposomes having a mean diameter of 0.01 microns to 3.0 microns, and wherein the platinum compound is cisplatin.

In a further embodiment the present invention relates to the aforementioned method, wherein the lipid is a mixture of DPPC and cholesterol in a 2 to 1 ratio by weight, the ratio of platinum compound to lipid in the lipid-based platinum compound formulation is 1:20 by weight, the lipid-based platinum compound formulation comprises liposomes having a mean diameter of 0.40 microns, and wherein the platinum compound is cisplatin.

In a further embodiment the present invention relates to the aforementioned method, wherein the patient is a human. In a further embodiment, the lipid-based platinum compound formulation is administered to the patient at least once every three weeks. In a further embodiment, the lipid-based platinum compound formulation is administered to the patient at least twice every three weeks. In a further embodiment, the lipid-based platinum compound formulation is administered to the patient at least three times every three weeks. In a further embodiment, the amount of platinum compound in the lipid-based platinum compound formulation is 60 mg/m² or greater, 100 mg/m² or greater, 140 mg/m² or greater, or 180 mg/m² or greater. In a further embodiment, the amount of platinum compound in the lipid-based platinum compound formulation is 100 mg/m² or greater, and the lipid-based platinum compound formulation is administered to the patient at least once every three weeks.

In another embodiment, the present invention relates to a lipid-based platinum compound formulation wherein the concentration of the platinum compound of the lipid-based platinum compound formulation is greater than about 1.2 mg/ml. In a further embodiment, the concentration of the platinum compound is about 3 mg/ml. In a further embodiment, the concentration of the platinum compound is about 5 mg/ml.

In another embodiment, the present invention relates to the aforementioned method, wherein the lipid-based platinum compound is prepared by (a) combining a platinum compound and a hydrophobic matrix carrying system; (b) establishing the mixture at a first temperature; (c) thereafter establishing the mixture at a second temperature, wherein the second temperature is cooler than the first temperature; and wherein the steps (b) and (c) are effective to increase the encapsulation of platinum compound. In a further embodiment the first temperature is from about 4° C. to about 70° C. In a further embodiment the second temperature is from about −25° C. to about 25° C. In a further embodiment the steps b) and c) are maintained for about 5 to 300 minutes.

These embodiments of the present invention, other embodiments, and their features and characteristics, will be apparent from the description, drawings and claims that follow.

### BRIEF DESCRIPTION OF THE DRAWINGS

FIG. 1 depicts the effect of intravenous treatment with lipid-based cisplatin (SLIT) vs. cisplatin on Lewis lung tumors in rats. Sprague Dawley rats were inoculated with Lewis lung carcinoma cells (2×10⁶) on day 0 and treated with bolus 6 mg/kg cisplatin or saline, cisplatin or lipid-based cisplatin on at 2 mg/kg on days 5, 8 and 11. Lungs were collected on day 15, observed and weighed and the tumor weight determined by normalization to the mean of historical control lung weights. Student T-test versus the saline data gave the p values shown.

FIG. 2 depicts tumor reduction in mice spleens after treatment with lipid-based cisplatin formulation: A) normal spleen; B) tumor bearing spleen treated by saline; and C) tumor bearing spleen treated by lipid-based cisplatin.

FIG. 3 depicts the higher amount of cisplatin from lipid-based cisplatin in the liver as compared to free cisplatin when administered intravenously.

FIG. 4 depicts the higher amount of cisplatin from lipid-based cisplatin in the lung as compared to free cisplatin when administered intravenously.

FIG. 5 depicts the increased amount of cisplatin from lipid-based cisplatin in the spleen when administered intravenously as compared to free cisplatin administered intravenously.

FIG. 6 depicts the increase in blood urea nitrogen (BUN) levels when free cisplatin is delivered either intravenously compared to lipid-based cisplatin administered by either method.

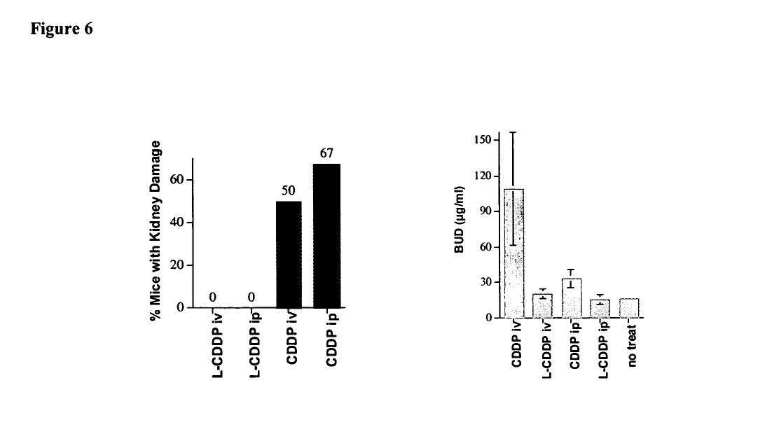

### DETAILED DESCRIPTION OF THE INVENTION

I. Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e. to at least one) of the grammatical object of the article. By way of example, “an element” means one element or more than one element.

The term “bioavailable” is art-recognized and refers to a form of the subject invention that allows for it, or a portion of the amount administered, to be absorbed by, incorporated to, or otherwise physiologically available to a subject or patient to whom it is administered.

The term “cancer treating effective amount” as used herein refers to the amount of lipid-based platinum compound formulation effective for the treatment of cancer. In one embodiment the cancer treating effective amount of lipid-based platinum compound formulation is typically about 100 mg/m² for ip delivery in a human.

The term “CDDP” stands for cis diamminedichloroplatinum, which is used interchangeably herein with “cisplatin”.

The terms “comprise” and “comprising” are used in the inclusive, open sense, meaning that additional elements may be included.

The term “hydrophobic matrix carrying system” is a lipid/solvent mixture prepared during the solvent infusion process described below.

The term “including” is used herein to mean “including but not limited to”. “Including” and “including but not limited to” are used interchangeably.

The term “intravenous” or “intravenously” or “iv” as used herein refers to administration of a therapeutic agent, such as, for example, an antineoplastic compound, such as a platinum compound, directly into the vascular system of a patient through a vein.

The term “L-CDDP” stands for a lipid-based formulation of cis diamminedichloroplatinum which is used interchangeably herein with “lipid-based cisplatin”.

The terms “lipid-based platinum compound” as used herein refers to a composition comprising a lipid and a platinum compound. In some embodiments the lipid-based platinum compound can be in the form of a liposome. In some embodiments the ratio of platinum compound to lipid in the lipid-based platinum compound can be between about 1:5 by weight and 1:50 by weight. In a further embodiment, the ratio of platinum compound to lipid in the lipid-based platinum compound can be between about 1:5 and about 1:30. In a further embodiment, the ratio of platinum compound to lipid in the lipid-based platinum compound can be between about 1:5 by weight and 1:25 by weight. In still other embodiments, the platinum compound can be cisplatin.

The term “mammal” is known in the art, and exemplary mammals include humans, primates, bovines, porcines, canines, felines, and rodents (e.g., mice and rats).

A “patient,” “subject” or “host” to be treated by the subject method may mean either a human or non-human animal.

The term “pharmaceutically-acceptable salts” is art-recognized and refers to the relatively non-toxic, inorganic and organic acid addition salts of compounds, including, for example, those contained in compositions of the present invention.

The term “solvent infusion” is a process that includes dissolving one or more lipids in a small, preferably minimal, amount of a process compatible solvent to form a lipid suspension or solution (preferably a solution) and then adding the solution to an aqueous medium containing bioactive agents. Typically a process compatible solvent is one that can be washed away in a aqueous process such as dialysis. The composition that is cool/warm cycled is preferably formed by solvent infusion. Alcohols are preferred as solvents, with ethanol being a preferred alcohol.

“Ethanol infusion,” is a type of solvent infusion that includes dissolving one or more lipids in a small, preferably minimal, amount of ethanol to form a lipid solution and then adding the solution to an aqueous medium containing bioactive agents. A “small” amount of solvent is an amount compatible with forming liposomes or lipid complexes in the infusion process.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

The term “therapeutic index” is an art-recognized term which refers to the ratio of a quantitative assessment of toxicity to a quantitative assessment of efficacy of a drug, e.g. LD₅₀/ED₅₀ in the case of animals. The term “LD₅₀” is art recognized and refers to the amount of a given toxic substance that will elicit a lethal response in 50% of the test organisms. This is sometimes also referred to as the median lethal dose. The term “ED₅₀” is art recognized and refers to the median effective dose.

The term “treating” is art-recognized and refers to curing as well as ameliorating at least one symptom of any condition or disease.

II. Introduction

Typical human parenteral dosage amounts with current commercial cisplatin solutions (e.g. Platinol from Bristol Myers Squibb) are about 100 mg/m² (unit dose) administered once every 3 weeks intravenously for a single drug therapy. Dose limiting factors include of course unwanted side effects (e.g. renal toxicity, severe vomiting . . . etc.). With higher potency platinum compound formulations, it would take less time to administer the platinum compound solution because less volume would have to be administered to achieve an equal amount of platinum compound. For example, a 100 mg/m² dosage with a human body surface of 1.6 to 2 m² (for the sake of this example, lets use 2 m²) would require administering 200 mg of platinum compound at one time. Using current commercial cisplatin solutions (1 mg/ml, the solubility limit at room temperature) requires infusing 200 ml of cisplatin solution into the body. At 5× the potency (5 mg/ml) only ⅕ the volume or 40 ml would need to be infused which also means the process would take ⅕ the amount of time.

Presented herein are platinum compound formulations that exceed their ordinary solubility limitations of 1 mg/ml and methods of treating cancer therewith. The greater potency of the platinum compound formulations is achieved by preparing the platinum compound formulations as lipid-based formulations. The high potency lipid-based platinum compound formulations of the present invention and methods of treating cancer therewith also benefit from the fact that the lipid-based formulations decrease the sub-acute toxicity of the platinum compound allowing even higher doses of platinum to be administered.

III. Lipids

The lipids used in forming the liposomes for iv delivery of a platinum compound may be synthetic, semi-synthetic or naturally-occurring lipids, including phospholipids, tocopherols, sterols, fatty acids, glycoproteins such as albumin, negatively-charged lipids and cationic lipids. In terms of phosholipids, they could include such lipids as egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the I position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include DPPC. Other examples include dimyristoylphosphatidycholine (DMPC) and dimyristoylphosphatidylglycerol (DMPG) dipalmitoylphosphatidcholine (DPPC) and dipalmitoylphosphatidylglycerol (DPPG) distearoylphosphatidylcholine (DSPC) and distearoylphosphatidylglycerol (DSPG), dioleylphosphatidyl-ethanolamine (DOPE) and mixed phospholipids like palmitoylstearoylphosphatidyl-choline (PSPC) and palmitoylstearolphosphatidylglycerol (PSPG), and single acylated phospholipids like mono-oleoyl-phosphatidylethanolamine (MOPE).

The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like.

The cationic lipids used can include ammonium salts of fatty acids, phospholids and glycerides. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP).

The negatively-charged lipids which can be used include phosphatidyl-glycerols (PGs), phosphatidic acids (PAs), phosphatidylinositols (PIs) and the phosphatidyl serines (PSs). Examples include DMPG, DPPG, DSPG, DMPA, DPPA, DSPA, DMPI, DPPI, DSPI, DMPS, DPPS and DSPS.

IV. Liposomes

Liposomes are completely closed lipid bilayer membranes containing an entrapped aqueous volume. Liposomes used for the parenteral delivery of a platinum compound may be unilamellar vesicles (possessing a single membrane bilayer) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes can be produced by a variety of methods (for a review, see, e.g., Cullis et al. (1987)). Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation.

Unilamellar vesicles can be produced from MLVs by a number of techniques, for example, the extrusion of Cullis et al. (U.S. Pat. No. 5,008,050) and Loughrey et al. (U.S. Pat. No. 5,059,421)). Sonication and homogenization cab be so used to produce smaller unilamellar liposomes from larger liposomes (see, for example, Paphadjopoulos et al. (1968); Deamer and Uster (1983); and Chapman et al. (1968)).

The original liposome preparation of Bangham et al. (J. Mol. Biol., 1965, 13:238-252) involves suspending phospholipids in an organic solvent which is then evaporated to dryness leaving a phospholipid film on the reaction vessel. Next, an appropriate amount of aqueous phase is added, the mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys, Acta., 1967, 135:624-638), and large unilamellar vesicles.

Techniques for producing large unilamellar vesicles (LUVs), such as, reverse phase evaporation, infusion procedures, and detergent dilution, can be used to produce liposomes. A review of these and other methods for producing liposomes may be found in the text Liposomes, Marc Ostro, ed., Marcel Dekker, Inc., New York, 1983, Chapter 1, the pertinent portions of which are incorporated herein by reference. See also Szoka, Jr. et al., (1980, Ann. Rev. Biophys. Bioeng., 9:467), the pertinent portions of which are also incorporated herein by reference.

Other techniques that are used to prepare vesicles include those that form reverse-phase evaporation vesicles (REV), Papahadjopoulos et al., U.S. Pat. No. 4,235,871. Another class of liposomes that may be used are those characterized as having substantially equal lamellar solute distribution. This class of liposomes is denominated as stable plurilamellar vesicles (SPLV) as defined in U.S. Pat. No. 4,522,803 to Lenk, et al. and includes monophasic vesicles as described in U.S. Pat. No. 4,588,578 to Fountain, et al. and frozen and thawed multilamellar vesicles (FATMLV) as described above.

A variety of sterols and their water soluble derivatives such as cholesterol hemisuccinate have been used to form liposomes; see specifically Janoff et al., U.S. Pat. No. 4,721,612, issued Jan. 26, 1988, entitled “Steroidal Liposomes.” Mayhew et al., PCT Publication No. WO 85/00968, published Mar. 14, 1985, described a method for reducing the toxicity of drugs by encapsulating them in liposomes comprising alpha-tocopherol and certain derivatives thereof. Also, a variety of tocopherols and their water soluble derivatives have been used to form liposomes, see Janoff et al., PCT Publication No. 87/02219, published Apr. 23, 1987, entitled “Alpha Tocopherol-Based Vesicles”.

Another method of preparing liposomes is the “solvent infusion” process. Solvent infusion is a process that includes dissolving one or more lipids in a small, preferably minimal, amount of a process compatible solvent to form a lipid suspension or solution (preferably a solution) and then adding the solution to an aqueous medium containing, for example, platinum compounds. Typically a process compatible solvent is one that can be washed away in an aqueous process such as dialysis. The composition that is cool/warm cycled is preferably formed by solvent infusion, with ethanol infusion being preferred.

The process for producing lipid-based platinum compound formulations may comprise mixing a platinum compound with an appropriate hydrophobic matrix and subjecting the mixture to one or more cycles of two separate temperatures. The process is believed to form active platinum compound associations.

In aqueous solution, when the platinum compound is cisplatin, it may form large insoluble aggregates with a diameter of greater than a few microns. In the presence of a amphipathic matrix system, such as a lipid bilayer, cisplatin-lipid associations form. For example, the associations may be formed in the internal aqueous space, the hydrocarbon core region of a lipid bilayer, or the liposome interface or headgroup. During the warming cycle of the process, it is believed that cisplatin is returned to solution at a greater rate in aqueous regions of the process mixture than from the lipid-matrix. As a result of applying more than one cool/warm cycle, cisplatin accumulates further from the lipid-matrix. Without limiting the invention to the proposed theory, experimentation indicates that the cisplatin-lipid associations cause the immediate surroundings of the interfacial bilayer region to be more hydrophobic and compact. This results in a high level of entrapment of active platinum compound as cooling and warming cycles are repeated.

The process comprises combining the platinum compound with a hydrophobic matrix carrying system and cycling the solution between a warmer and a cooler temperature. Preferably the cycling is performed more than one time. More preferably the step is performed two or more times, or three or more times. The cooler temperature portion of cycle can, for example, use a temperature from about −25° C. to about 25° C. More preferably the step uses a temperature from about −5° C. to about 25° C. or from about 1° C. to about 20° C. For manufacturing convenience, and to be sure the desired temperature is established, the cooler and warmer steps can be maintained for a period of time, such as approximately from 5 to 300 minutes or 30 to 60 minutes. The step of warming comprises warming the reaction vessel to from about 4° C. to about 70° C. More preferably the step of warming comprises heating the reaction vessel to about 45° C. or to about 55° C. The above temperature ranges are particularly preferred for use with lipid compositions comprising predominantly diphosphatidycholine (DPPC) and cholesterol.

Another way to consider the temperature cycling is in terms of the temperature differential between the warmer and the cooler steps of the cycle. This temperature differential can be, for example, about 25° C. or more, such as a differential from about 25° C. to about 70° C., preferably a differential from about 40° C. to about 55° C. The temperatures of the cooler and higher temperature steps are selected on the basis of increasing entrapment of active platinum compound. Without being limited to theory, it is believed that it is useful to select an upper temperature effective substantially increase the solubility of active platinum compound in the processed mixture. Preferably, the warm step temperature is about 50° C. or higher. The temperatures can also be selected to be below and above the transition temperature for a lipid in the lipid composition.

The temperatures appropriate for the method may, in some cases, vary with the lipid composition used in the method, as can be determined by ordinary experimentation.

The platinum compound to lipid ratio seen in the lipid-based platinum formulations used in the present invention may be between about 1:5 by weight and about 1:50 by weight. More preferably the platinum compound to lipid ratio achieved is between about 1:5 by weight and about 1:30 by weight. Most preferably the platinum compound to lipid ratio achieved is between about 1:5 by weight and about 1:25 by weight.

The liposomes have a mean diameter of approximately 0.01 microns to approximately 3.0 microns, preferably in the range about 0.1 to 1.0 microns. More preferably, the mean diameter is from about 0.2 to 0.5 microns. The sustained release property of the liposomal product can be regulated by the nature of the lipid membrane and by inclusion of other excipients (e.g., sterols) in the composition.

In a preferred embodiment of the invention the liposome contains about 50 to about 100 mol % DPPC and about 0 to about 50 mol % cholesterol. More preferably, the liposome contains about 50 to about 65 mol % DPPC and about 35 to about 50 mol % cholesterol.

Liposomes can also be prepared by the methods disclosed in copending U.S. patent application Ser. No. 10/383,004, filed Mar. 5, 2003; Ser. No. 10/634,144, filed Aug. 4, 2003; Ser. No. 10/224,293, filed Aug. 20, 2002; and Ser. No. 10/696,389, filed Oct. 29, 2003, the specifications of which are incorporated herein in their entirety.

V. Platinum Compounds

The platinum compounds that may be used in the present invention include any compound that exhibits the property of preventing the development, maturation, or spread of neoplastic cells. Non-limiting examples of platinum compounds include cisplatin, carboplatin (diammine(1,1-cyclobutanedicarboxylato)-platinum(II)), tetraplatin (ormaplatin) (tetrachloro(1,2-cyclohexanediamine-N,N′)-platinum(IV)), thioplatin (bis(O-ethyldithiocarbonato)platinum(II)), satraplatin, nedaplatin, oxaliplatin, heptaplatin, iproplatin, transplatin, lobaplatin, cis-aminedichloro(2-methylpyridine) platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine) platinum(IV)), JM335 (trans-amminedichloro(cyclohexylamine)dihydroxoplatinum(IV)), and (trans, trans, trans)bis-mu-(hexane-1,6-diamine)-mu-[diamine-platinum(II)]bis[diamine(chloro) platinum(II)]tetrachloride. In another embodiment the platinum compound is cisplatin. Depending on the environment, cisplatin may exist in a cationic, aquated form wherein the two negatively charged chloride atoms have been displaced by two neutral water molecules. Because the aquated form of cisplatin is cationic, anionic lipids such as glycerols help to stabilize the lipid-based formulation, but may also hinder release on the cisplatin. The non-aquated, neutral form of cisplatin is more difficult to stabilize but has different release kinetics. It is considered an advantage of the present invention that in certain embodiments the lipid-based cisplatin formulations comprise neutral cisplatin and neutral lipids. Because of the equilibrium between neutral, non-aquated cisplatin and cationic, aquated cisplatin, one may favor neutral, non-aquated cisplatin by preparing a formulation with a low pH and high NaCl concentration. In this embodiment a substantial amount of the cationic, aquated form of cisplatin would not form until the neutral, non-aquated cisplatin was delivered into the interior of a cell.

In other embodiments, other therapeutic agents may be used with the platinum compounds. The other therapeutic agents may have antineoplastic properties. Non-limiting examples of antineoplastic compounds include altretamine, amethopterin, amrubicin, annamycin, arsenic trioxide, asparaginase, BCG, benzylguanine, bisantrene, bleomycin sulfate, busulfan carmustine, cachectin, chlorabucil, 2-chlorodeoxyadenosine, cyclophosphamide, cytosine arabinoside, dacarbazine imidazole carboxamide, dactinomycin, daunomycin, 3′-deamino-3′-morpholino-1,3-deoxo-10-hydroxycarminomycin, 4-demethoxy-3-deamino-3-aziridinyl-4-methylsulphonyl-daunorubicin, dexifosfamide, dexamethasone, diarizidinylspermine, dibromodulcitol, dibrospidium chloride, 1-(11-dodecylamino-10-hydroxyundecyl)-3,7-dimethylxanthine, doxorubicin, elinafide, epipodophyllotoxin, estramustine, floxuridine, fluorouracil, fluoxymestero, flutamide, fludarabine, fotemustine, galarubicin, glufosfamide, goserelin, GPX100, hydroxyurea, idarubicin HCL, ifosfamide, improsulfan tosilate, isophosphamide, interferon alfa, interferon alfa 2a, interferon alfa 2b, interferon alfa n3, interferon gamma, interleukin 2, irinotecan, irofulven, leucovorin calcium, leuprolide, levamisole, lomustine, megestrol, L-phenylalanie mustard, L-sarcolysin, melphalan hydrochloride, mechlorethamine, MEN10755, mercaptopurine, MESNA, methylprednisolone, methotrexate, mitomycin, mitomycin-C, mitoxantrone, nimustine, paclitaxel, pinafide, pirarubicin, plicamycin, prednimustine, prednisone, procarbazine, profiromycin, pumitepa, ranimuistine, sertenef, streptozocin, streptozotocin, tamoxifen, tasonermin, temozolomide, 6-thioguanine, thiotepa, tirapazimine, triethylene thiophosphoramide, trofosfamide, tumor necrosis factor, valrubicin, vinblastine, vincristine, vinorelbine tartrate, and zorubicin.

Also included as suitable platinum compounds used in the methods of the present invention are pharmaceutically acceptable addition salts and complexes of platinum compounds. In cases wherein the compounds may have one or more chiral centers, unless specified, the present invention comprises each unique racemic compound, as well as each unique nonracemic compound.

In cases in which the platinum compounds have unsaturated carbon-carbon double bonds, both the cis (Z) and trans (E) isomers are within the scope of this invention. In cases wherein the neoplastic compounds may exist in tautomeric forms, such as keto-enol tautomers, such as 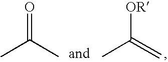
each tautomeric form is contemplated as being included within this invention, whether existing in equilibrium or locked in one form by appropriate substitution with R′. The meaning of any substitutent at any one occurrence is independent of its meaning, or any other substitutent's meaning, at any other occurrence.

Also included as suitable platinum compounds used in the methods of the present invention are prodrugs of the platinum compounds. Prodrugs are considered to be any covalently bonded carriers which release the active parent compound in vivo.

The present invention, in part, discloses methods of treating cancer more effectively which may have lower nephrotoxicity previously not disclosed. By using lipid-based formulations and ip delivery, a more potent and efficient cancer treatment is achieved.

VI. Dosages

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 50 mg per kg.

Dosage amounts are also commonly administered as mg/m² which stands for milligrams of drug (e.g. platinum compound) per body surface area. Generally, dosage amounts for platinum compounds may be about 60 mg/m² or greater, 100 mg/m² or greater, 140 mg/m² or greater, or 180 mg/m² or greater. Dosage amounts of about 140 mg/m² or greater are generally considered at the high end of tolerance, but an advantage of the present invention is that the platinum compound is administered as part of a lipid-based formulation which decreases the sub-acute toxicities of the platinum compound. It is therefore envisioned by the inventors that higher than normal dosage amounts of platinum compound may be administered to the patient without unwanted toxic side effects. Higher dosages may lead to longer duration cycles between dosages and greater convenience for the patient. For example, dosage amounts are generally administered to the patient once about every three weeks. If higher dosage amounts of platinum compound can be administered safely to the patient then the cycle time may be increased to once about every four, five, six, seven, or even eight weeks. Longer cycle times means less trips to a care facility for treatment and less times the patient would have to undergo the administration process.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions (e.g., the antineoplastic compound) because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

VII. Pharmaceutical Formulation

The pharmaceutical formulation of the antineoplastic compound may be comprised of an aqueous dispersion of liposomes. The formulation may contain lipid excipients to form the liposomes, and salts/buffers to provide the appropriate osmolarity and pH. The pharmaceutical excipient may be a liquid, diluent, solvent or encapsulating material, involved in carrying or transporting any subject composition or component thereof from one organ, or portion of the body, to another organ, or portion of the body. Each excipient must be “acceptable” in the sense of being compatible with the subject composition and its components and not injurious to the patient. Suitable excipients include trehalose, raffinose, mannitol, sucrose, leucine, trileucine, and calcium chloride. Examples of other suitable excipients include (1) sugars, such as lactose, and glucose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### EXEMPLIFICATION

### Example 1

Method of Producing an Aqueous Cisplatin with Higher Potency than its Aqueous Solubility Limit at Room Temperature.
1) At temperatures about 50-60° C., cisplatin in 0.9% sodium chloride solution at a level of 4 mg/ml and an ethanolic solution of about 16 mg/ml DPPC and 8 mg/ml cholesterol at about 55° C. are aseptically prepared.
2) The lipid solution is infused into the cisplatin solution while mixing the cisplatin solution.
3) After infusion, cisplatin/lipid dispersion is cooled down to about 10° C. and then warmed up again to about 50-60° C. for 15 min.
4) Step 3) is repeated 2-3 times.
5) The dispersion is aseptically washed with sterile 0.9% sodium chloride solution to remove residual ethanol and un-associated cisplatin via 500,000 MW cut-off membrane diafiltration unit.

After washing process, the dispersion provides about 1 mg/ml cisplatin potency and concentrated to 3 mg/ml cisplatin and further concentrated to 5 mg/ml cisplatin by aseptically removing two third of the aqueous vehicle of 1 mg/ml product and four fifth of the aqueous vehicle of 1 mg/ml product, respectively. The removal of aqueous vehicle was carried out at a rate of about 100 ml/min by diafiltration at about 20° C. without compensating the permeate with fresh sterile 0.9% sodium chloride solution.

### Example 2

Comparison of preclinical in-vivo antitumor activity of inhaled and iv injected lipid-based cisplatin formulations in a murine Lewis lung tumor metastasis model. C57B1/6 mice (Charles Rivers) were ear-tagged upon arrival at Transave vivarium (Procedure #1(P-1). Ninety-seven (97) mice were injected with 2×10⁶ Lewis Lung cells on Day 0 (P-26). Eighty-four mice survived the injection of tumor cells. On day 1, the mice were weighed (P-14) and randomized (P-31) into 7 groups of 12 mice and one group of 3 mice. On days 5, 8, and 11, mice in group 1 were treated with nebulized 0.9% saline (Abbott) using LC Star nebulizer (Pari), Proneb Ultra and Proneb Turbo compressors (Pari) and 12 port nose-only chamber (CH Technologies) for 60 min for each session (P-22). On days 5, 8, and 11, mice in group 2 were treated with nebulized Slit-Cisplatin (1 mg/ml, DVLP-CISP-3L-06A) using LC Star nebulizer (Pari), Proneb Ultra and Proneb Turbo compressors (Pari) and 12 port nose-only chamber (CH Technologies) for 60 min for each session (P-22). On days 5, 8, and 11, mice in group 3 received iv injections (P-26) of Slit-cisplatin (2 mg/kg). On day 5, mice in group 4 received a single bolus iv injection of cisplatin (6 mg/kg). On days 5, 8, and 11, mice in group 5 were treated with nebulized Cisplatin (1 mg/ml, Baxter) using LC Star nebulizer (Pari), Proneb Ultra and Proneb Turbo compressors (Pari) and 12 port nose-only chamber (CH Technologies) for 30 min for each session (P-22). On days 5, 8, and 11, mice in group 6 received iv injections of soluble cisplatin (2 mg/kg). On day 5, mice in group 7 received a single bolus iv injection of saline.

On day 15, all mice were euthanized (P-12). Their lungs were removed and weighed (Ohaus Analytical Plus Balance) (P-30). The tumor weigh of each mouse was calculated by subtracting the median (0.177 mg) lung weight of control mice that were not injected with tumor cells. Approximately 20% of the mice in the saline group failed to have lung weights greater than the control lung weight suggesting that tumors were never established in their lungs. Therefore these lungs weights and similar lung weights from each group were also eliminated from the analysis since the mice were randomized after the injection of LL cells. Therefore it would be expected that approximately 20% of the mice in each group would have failed to establish tumors in their lungs.

Blood and urine were collected (P-21) from mice after they were euthanized. Plasma was separated from EDTA (20 mM) blood by centrifugation and stored at −80° C. Urine was also stored at −80° C. Plasma and urine were analyzed for creatinine concentration utilizing a commercial creatinine assay kit (Cayman Chemical Co).

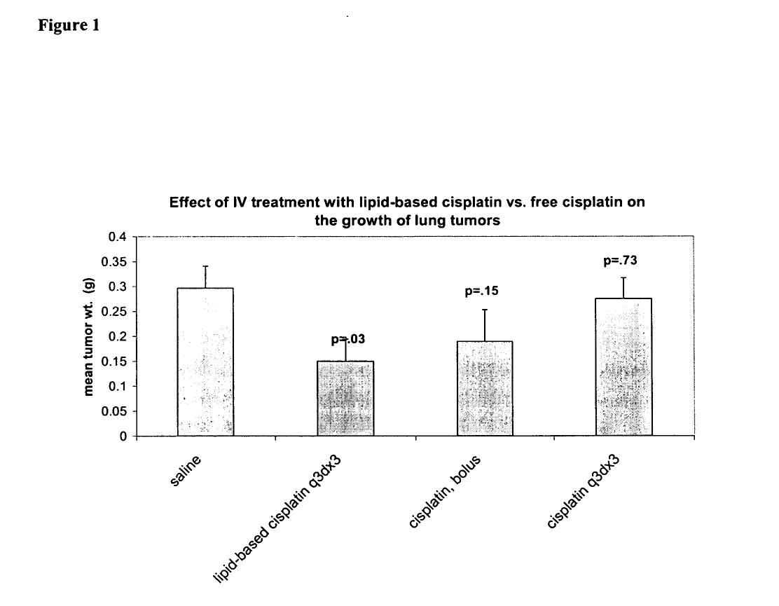

Statistical analysis (Student t-test) of the lung tumor weighs and creatinine concentrations was performed using the Excel from Microsoft. The tumor weights of each group are depicted in FIG. 1. The p values are a comparison of each group to the saline control group.

### Example 3

In vivo effect of iv administered lipid-based cisplatin formulation on MCA 38 Adenocarcinoma Cells. MCA 38 Adenocarcinoma Cells (1×10⁶/50 ul) were injected into the spleens of 20 anesthetized female C57B16 mice. Four (4) days later the mice were randomized into 2 groups of 10 mice per groups. The first group of mice received iv injection 100 ul of saline on days 4, 7, and 10 post injections of MCA38 cells. The second group received iv injection of 2 mg/kg of lipid-based cisplatin formulation (1 mg/ml) on days 4, 7, and 10. On day 18 the mice were euthanized with CO₂. Their bodies, spleens and livers were weighed and recorded. Their livers were fixed in 10% buffered formalin overnight then washed in water the following day. The livers were stained with Putt's Alcian Blue method to identify the liver metastases on the surface of the liver. The metastases were counted using a dissecting microscope (20× magnification). Lipid-based cisplatin administered intravenously substantially reduced number of metastases on mice liver as shown in Table 1.

TABLE 1
Mice liver metastases results.

| | Treatment | |
|---|---|---|
| | Saline (control) | Lipid-based cisplatin |
| Number of metastases | 6.6 ± 5.5 per mouse | 1.6 ± 1.8 per mouse |

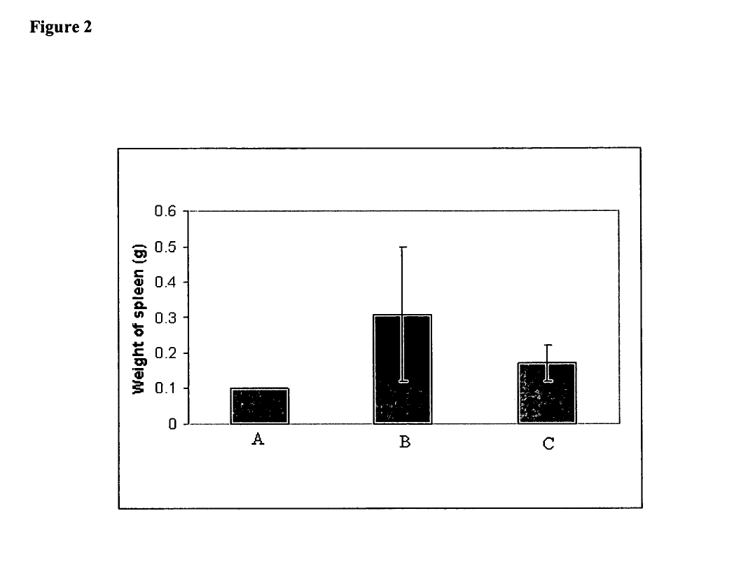

Spleen results are presented in FIG. 2.

### Example 4

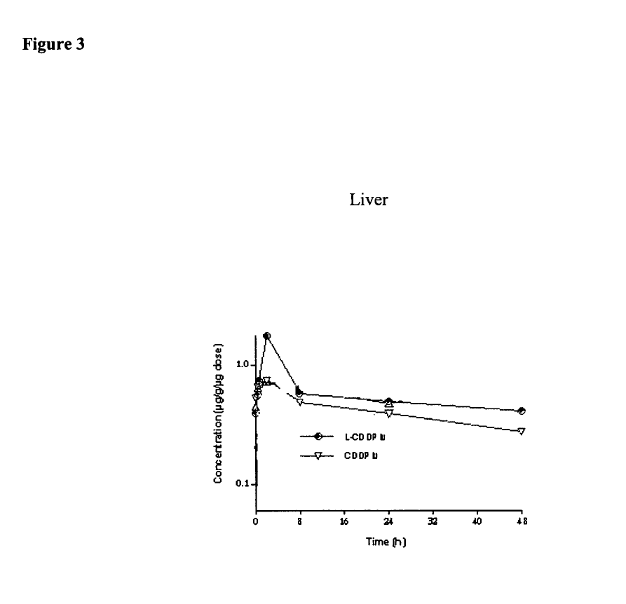

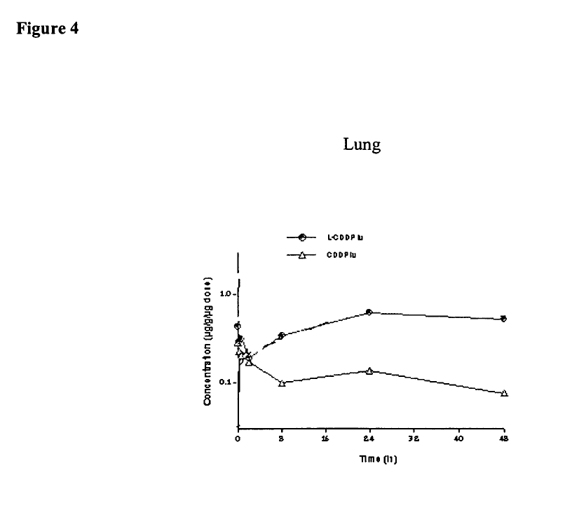

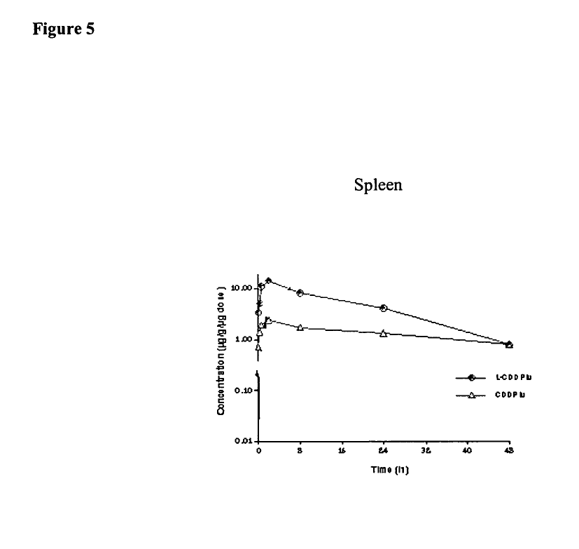

Pharmacokinetics and organ distribution in animals of ip and iv injected lipid-based cisplatin and cisplatin (Part II). Sixty ICR mice (female, 7 weeks old) were divided into 4 groups. They received intraperitoneal or intravenous injection of L-CDDP or CDDP, separately. The dose was 12 mg/kg for ip L-CDDP and 8 mg/kg for the rest of treatment groups. At each designed time point, three to four mice were anaesthetized with 70 mg/kg of Nembutal ip (e.g., 3, 20, and 40 min, and 2, 8, 24, 48, and 72 h). The blood was drawn from the inferior vena cava. Organs including liver, lung, and spleen were resected from the mice. The organ samples were homogenized in distilled water (4-fold of the sample weight) and digested with nitric acid. The platinum concentration in each sample was measured by Inductively Coupled Plasma-Mass Spectrometer (ICP-MS). The pharmacokinetics profiles (FIGS. 3, 4, and 5, all Y-axes are concentration of μg platinum in one gram of tissue or fluid per mg of injected dose) and parameters (Table 2) of each formulation were simulated and calculated. It was found that 1) uptake of platinum in the spleen was significantly enhanced by the liposomal formulation irrespective of the injection routes, the AUCs and Cₘₐₓ were 26˜47-fold higher than those of CDDP, p<0.0003; 2) with ip injection, the circulation AUC and elimination t_{1/2} of L-CDDP were 4- and 15-fold (p<0.006) higher than those of CDDP; 3) the lung and liver platinum uptake of iv L-CDDP were higher than that of iv CDDP (Lung: p=0.043; Liver: p=0.051).

TABLE 2
Organ AUC and Cₘₐₓ.

| iv | |
|---|---|
| AUC (h μg/g/μg) | Cₘₐₓ (μg/g/μg) |

| | L-CDDP | CDDP | L-CDDP | CDDP |
|---|---|---|---|---|
| Liver | 28.22 | 19.96 | 1.74 | 0.74 |
| Lung | 23.30^{d} | 5.58 | 0.61 | 0.28 |
| Spleen | 241.38^{g} | 8.11 | 13.80^{h} | 0.29 |

Two side Log rank test was used to evaluate the significance of the differences of L-CDDP versus CDDP in AUC or Cₘₐₓ values. Statistically significant pairs (p < 0.05) were labeled with a superscript letter. Their p values are as follows:
^{c} p = 0.046;
^{d}p = 0.008;
^{e} p = 0.0002;
^{f} p = 0.0001;
^{g}p = 0.0003;
^{h}p = 0.0002.

### Example 5

Nephrotoxicity. ICR mice, 7 weeks old, female, were divided into 4 groups with 3 to 4 mice in each. They were injected with maximum tolerated dose (MTD) of L-CDDP or CDDP via iv or ip. Four days after the injection, the mice were euthanized with Nembutal ip. The blood was drawn and the serum was isolated. The blood urea nitrogen (BUN) was quantitatively measured with a colorimetric method at Antech Diagnostics. Organs including duodenum, heart, kidney, liver, lung, and spleen were resected from the mice and fixed with 10% buffered Formalin. The fixed tissues were processed with standard procedure for H and E staining. A pathology expert Dr. Carman Tomos at the Memorial Sloan-Kettering Cancer Center examined kidney tissues and gave a toxicity grade to each kidney tissue sample. The grading was based on the general pathology guidelines for kidney toxicity.

The pathological results demonstrate that irrespective of administration routes, CDDP caused severe nephrotoxicity in more than 50% mice receiving the treatment, but L-CDDP did not cause any nephrotoxicity. The similar conclusion can be drawn from BUN test (FIG. 8), where iv CDDP significantly increased BUN level by 6,8-fold compared with normal controls (p=0.008), and ip CDDP caused much less BUN accumulation, only 2,1-fold increase compared to normal controls (p=0.04), but L-CDDP injected by either route did not cause BUN level elevation.

### Incorporation by Reference

All of the patents and publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of treating cancer in a patient comprising administering intravenously to a patient in need thereof a cancer treating effective amount of a lipid-based platinum compound formulation wherein the concentration of the platinum compound of the lipid-based platinum compound formulation is greater than about 1.2 mg/ml.

2. The method of claim 1, wherein the platinum compound concentration is about 3 mg/ml.

3. The method of claim 1, wherein the platinum compound concentration is about 5 mg/ml.

4. The method of claim 1, wherein the platinum compound is selected from the group consisting of: cisplatin, carboplatin (diammine(1,1-cyclobutanedicarboxylato)-platinum(II)), tetraplatin (ormaplatin) (tetrachloro(1,2-cyclohexanediamine-N,N′)-platinum(IV)), thioplatin (bis(O-ethyldithiocarbonato)platinum(II)), satraplatin, nedaplatin, oxaliplatin, heptaplatin, iproplatin, transplatin, lobaplatin, cis-aminedichloro(2-methylpyridine) platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine) platinum(IV)), JM335 (trans-amminedichloro (cyclohexylamine)dihydroxoplatinum(IV)), (trans, trans, trans)bis-mu-(hexane-1,6-diamine)-mu-[diamine-platinum(II)]bis[diamine(chloro) platinum(II)]tetrachloride, and mixture thereof.

5. The method of claim 1, wherein the platinum compound is cisplatin.

6. The method of claim 1, wherein the lipid in the lipid-based platinum compound formulation is comprised of a member selected from the group consisting of: egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidyl choline (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (PIs), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof.

7. The method of claim 1, wherein the lipid in the lipid-based platinum compound formulation is a mixture of a phospholipid and a sterol.

8. The method of claim 1, wherein the lipid in the lipid-based platinum compound formulation is a mixture of DPPC and cholesterol.

9. The method of claim 1, wherein the lipid in the lipid-based platinum compound formulation is a mixture of DPPC from 50 to 65 mol % and cholesterol from 35 to 50 mol %.

10. The method of claim 1, wherein the cancer is selected from the following: melanoma, testis (germ cell), osteosarcoma, soft tissue sarcoma, thyroid cancer, colon cancer, ovarian cancer, cancer of the kidney, breast cancer, colorectal cancer, prostate cancer, bladder cancer, uterine cancer, lung cancer, stomach cancer, liver cancer, spleen cancer, endometrial, or squamous cell carcinomas of the head and neck.

11. The method of claim 1, wherein the cancer is lung cancer.

12. The method of claim 1, wherein the cancer is spleen cancer.

13. The method of claim 1, wherein the cancer is liver cancer.

14. The method of claim 1, wherein the ratio of platinum compound to lipid in the lipid-based platinum compound formulation is between 1:5 by weight and 1:50 by weight.

15. The method of claim 1, wherein the lipid-based platinum compound formulation comprises liposomes having a mean diameter of 0.01 microns to 3.0 microns.

16. The method of claim 1, wherein the lipid is a mixture of DPPC and cholesterol, the ratio of platinum compound to lipid in the lipid-based platinum compound formulation is between 1:5 by weight and 1:50 by weight, and wherein the lipid-based platinum compound formulation comprises liposomes having a mean diameter of 0.01 microns to 3.0 microns.

17. The method of claim 1, wherein the lipid is a mixture of DPPC and cholesterol, the ratio of platinum compound to lipid in the lipid-based platinum compound formulation is between 1:5 by weight and 1:50 by weight, the lipid-based platinum compound formulation comprises liposomes having a mean diameter of 0.01 microns to 3.0 microns, and wherein the platinum compound is cisplatin.

18. The method of claim 1, wherein the lipid is a mixture of DPPC and cholesterol in a 2 to I ratio by weight, the ratio of platinum compound to lipid in the lipid-based platinum compound formulation is about 1:20 by weight, the lipid-based platinum compound formulation comprises liposomes having a mean diameter of about 0.40 microns, and wherein the platinum compound is cisplatin.

19. The method of claim 1, wherein the patient is a human.

20. The method of claim 1, wherein the lipid-based platinum compound formulation is administered to the patient at least once every three weeks.

21. The method of claim 1, wherein the amount of platinum compound in the lipid-based platinum compound formulation is 60 mg/m² or greater, 100 mg/m² or greater, 140 mg/m² or greater, or 180 mg/m² or greater.

22. The method of claim 1, wherein the amount of platinum compound in the lipid-based platinum compound formulation is 100 mg/m² or greater, and the lipid-based platinum compound formulation is administered to the patient at least once every three weeks.
